(19) United States
(12) Patent Application Publication
(10) Pub. No.: US 2005/0027173 A1
(43) Pub. Date: Feb. 3, 2005

(54) Brain injury protocols

(76) Inventors: Kathleen E. Briscoe, Carnation, WA (US); Michael D. McMahon, Lake Forest Park, WA (US); Stephen W. Radons, Snohomish, WA (US); Joseph L. Sullivan, Kirkland, WA (US)

Correspondence Address: SHUMAKER & SIEFFERT, P. A., 8425 SEASONS PARKWAY, SUITE 105, ST. PAUL, MN, 55125, US

(21) Appl. No.: 10/632,121

(22) Filed: Jul. 31, 2003

(51) Int. Cl. A61B 5/00; G06F 17/60

(52) U.S. Cl. 600/300; 128/920; 604/66; 607/96

(57) ABSTRACT

In general, the invention is directed to techniques for managing health care protocols, and in particular, brain injury protocols, with a system that may be incorporated with a device such as a defibrillator, patient monitor, or other device. The system selects a brain injury protocol as a function of patient data received from an operator, or from a monitoring device, or both. Pursuant to the protocol, the system may present information, direct an operator to perform a task, determine a presumptive diagnosis, and control a therapy device.

### TECHNICAL FIELD

The invention relates to patient health care protocols, and more particularly, to protocols implemented with the assistance of electronic devices.

### BACKGROUND

Emergency medical technicians (EMTs) save lives every day by responding to emergencies. EMTs provide immediate medical attention to a patient. Medical attention may include, for example, determining the nature and extent of the condition of the patient and administering therapy.

Jurisdictions generally recognize degrees of proficiency among EMTs. Some EMTs are trained and qualified to provide an extensive range of pre-hospital care, and others are trained and qualified to provide lesser degrees of care. Various jurisdictions hold EMTs subject to strict rules and guidelines pertaining to appropriate emergency care. The rules and guidelines differ from jurisdiction to jurisdiction.

EMTs often respond to emergencies involving brain injury, such as brain injury resulting from cardiac arrest, transient ischemic attacks, ischemic stroke, hemorrhagic stroke, and closed head trauma. EMTs routinely apply one or more recognized procedures for assessing the neurological condition of the patient by observing the appearance and demeanor of the patient, and the responses of the patient to stimuli.

EMTs are not the only individuals who address such emergencies. Physicians, nurses and other emergency workers may address them as well. Physicians in a hospital setting, for example, may apply the same recognized procedures to evaluate the neurological condition of the patient as are applied by an EMT in the field.

### SUMMARY

In general, the invention is directed to techniques for managing health care protocols, and in particular, brain injury protocols, with a system that may be incorporated with a device such as a defibrillator, patient monitor, or other device. The system may be brought to the site of a patient in need of medical assistance. The system selects a brain injury protocol as a function of patient data received from an operator, or from a monitoring device, or both. Pursuant to the protocol, the system may present information, direct an operator to perform a task, determine a presumptive diagnosis, and control a therapy device.

In a typical application involving an actual or suspected brain injury, the patient may be unconscious or dysfunctional. Accordingly, a person other than the patient serves as an operator for the system. In some circumstances, however, the patient may be the operator. For purposes of simplicity, the patient and the operator will be described herein as separate people.

In general, the term “brain injury protocols” encompasses procedures for examining, evaluating and treating patients with actual or suspected brain injury. Pursuant to a protocol, the complaint, condition or presentation of a patient, and the response of the patient to stimuli may be observed and recorded. For example, the operator may administer, pursuant to a brain injury protocol, any of several recognized procedures for assessing the neurological condition of a patient, such as the Cincinnati Prehospital Stroke Scale, the Los Angeles Prehospital Stroke Screen, or the Glasgow Coma Scale.

The system may collect patient data from the patient via a monitoring device such as a blood pressure cuff, temperature sensor, blood oxygen meter, and the like. The system may also collect data from the operator via one or more input devices, such as a touch screen, a button or a pointing tool. Based upon the patient data, the system selects a brain injury protocol. The system may select a new brain injury protocol as more data are collected. For example, the system may select a general stroke protocol, and later select a more specific protocol pertaining to ischemic stroke.

In one embodiment, the invention is directed to a method comprising receiving data concerning a patient and selecting a brain injury protocol as a function of the data. The data may be received from an operator, a monitoring device, or both. The method may further comprise controlling a therapy device according to the brain injury protocol, or presenting an operator with a task to be performed pursuant to the brain injury protocol.

In another embodiment, the invention is directed to a method comprising presenting a checklist to an operator, receiving the data from the operator in response to the checklist, receiving additional data concerning the patient from a monitoring device, and selecting a brain injury protocol as a function of the data. In general, the checklist requests entry of data concerning a neurological condition of a patient, and may include one or more of the Cincinnati Prehospital Stroke Scale, the Los Angeles Prehospital Stroke Screen, the Glasgow Coma Scale and the Hunt and Hess Scale for Subarachnoid Hemorrhage.

In further embodiments, the invention is directed to a computer-readable medium containing instructions for causing a programmable processor to carry out the methods described above.

In an additional embodiment, the invention is directed to a system comprising a monitoring device to monitor a medical condition of a patient and to generate data concerning the patient, and a processor to receive the data from the monitoring device and to select a brain injury protocol as a function of the data. The system may also include a therapy device to provide brain injury therapy to the patient, and may include an input device to receive patient data from an operator.

Various embodiments of the invention may offer one or more advantages, which will be described in detail below. In general, the invention helps guide the medical personnel through the established procedures of an applicable protocol, helps the emergency medical personnel apply the appropriate procedures, and records events pertaining to examination, evaluation and treatment of the patient. The invention may also provide a convenient integration of functions that are traditionally separate.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic view of a system that implements the techniques of the invention.

FIG. 2 is a flow diagram illustrating a technique for selection of a brain injury protocol, presentation of information pursuant to the selected brain injury protocol, and control of a therapy device according to an embodiment of the invention.

FIG. 3 is an example of a screen display that lists neurological examination brain injury protocols, according to an embodiment of the invention.

FIG. 4 is an example of a screen display showing a presumptive diagnosis, according to an embodiment of the invention.

FIG. 5 is an example of a screen display of an event log, according to an embodiment of the invention.

FIG. 6 is an example of a screen display that presents information pursuant to a brain injury protocol and receives patient data, according to an embodiment of the invention.

FIG. 7 is an example of a screen display that presents information pursuant to a brain injury protocol and receives data, according to an embodiment of the invention.

### DETAILED DESCRIPTION

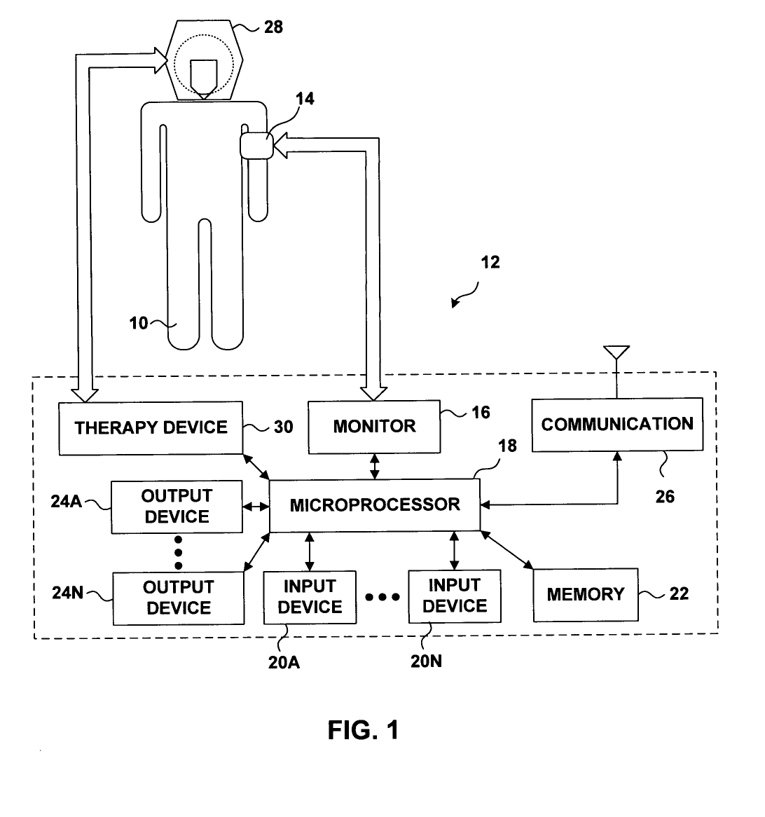

FIG. 1 is a block diagram showing a patient 10 coupled to an exemplary system 12 according to an embodiment of the invention. System 12 is shown in FIG. 1 as incorporated with a monitoring device, but system 12 may be incorporated with other devices as well, such as an automated external defibrillator (AED) or a patient cooling apparatus.

System 12 includes a monitoring device 14, which is coupled to system 12 via a monitoring device interface 16. Monitoring device 14 may be any device that detects, monitors or measures any characteristic of patient 10. Monitoring device 14 may be, but need not be, proximate to patient 10 or in contact with patient 10. In FIG. 1, monitoring device 14 is depicted as a blood pressure cuff, but monitoring device 14 may be any of several monitoring devices, such as a temperature sensor, a blood oxygen meter, a carbon dioxide sensor, a heart rate sensor, a pulse detector, a respiration sensor, a blood velocity sensor, an electroencephalogram (EEG) and the like. A multi-lead set of electrodes, such as a twelve-lead apparatus for sensing an electrocardiogram, is also an example of a monitoring device, as is an electrode pair, such as the electrodes of an AED.

Microprocessor 18 receives and process data collected via monitoring device 14. Microprocessor 18 selects a brain injury protocol as a function of the data. In addition, microprocessor 18 receives data from an operator via one or more input devices 20A-20N (hereinafter 20), such as one or more buttons, a keyboard, a touch screen, a voice recognition module or a pointing tool. Microprocessor 18 selects a brain injury protocol as a function of the data received from the operator.

The operator may enter any data concerning patient 10 via input device 20. The operator may, for example, enter information pertaining to the physical condition of patient 10, the mental condition of patient 10 and a complaint from patient 10. The operator may be prompted to enter information about the age, sex, race, height or weight of patient 10. Patient data may also include the medical history of patient 10, such as history of factors that may make patient 10 susceptible to stroke.

“Brain injury protocol,” as used herein, is defined broadly. “Brain injury protocol” encompasses procedures for examining, evaluating and treating patients with actual or suspected brain injury. The procedures may include plans, guidelines and rules for treating patients, and are stored in a memory unit 22. Memory 22 may include volatile storage, such as random access memory, and/or non-volatile storage, such as Flash memory or a hard disk. The term “brain injury protocol” encompasses general procedures, as well as procedures applicable to a specific patient complaint, condition or presentation. “Brain injury protocol” further includes rules and guidelines applicable to a jurisdiction, such as treatment procedures adopted by a regulating authority responsible for overseeing EMTs. A regulating authority may be, for example, a regional health care system administrator or a government administrative agency that licenses and regulates EMTs.

Different regulating authorities may require different brain injury protocols. In a typical application, memory 22 stores the brain injury protocol for the jurisdiction in which system 12 is used. Memory 22 may also store other brain injury protocols as well. Memory 22 may further store protocols in addition to brain injury protocols, such as defibrillation therapy protocols, or protocols pertaining to trauma such as bleeding, broken bones or burns.

The procedures of a brain injury protocol may be embodied as a checklist, a questionnaire, a flow diagram, a series of notices or the like. As used herein, “brain injury protocol” includes the embodiment of the procedures, as well as what an operator should do in carrying out the procedures. Some brain injury protocols include diagnosis. For example, a brain injury protocol may involve collecting information about the exhibited symptoms, complaints, medical history and vital signs of the patient. Microprocessor 18 collects the information via one or more monitoring devices 14, or via operator input via one or more input devices 20, or both, and can select a more specific brain injury protocol. The selected brain injury protocol may direct that the patient will be suspected of suffering from a particular condition, and will be treated accordingly. Another brain injury protocol may pertain to the diagnosed condition or the treatment for the diagnosed condition.

As used herein, “brain injury protocol” also includes sub-protocols that may be used in comprehensive protocols. A sub-protocol pertaining to the application of leads to measure an ECG, for example, may be included in a brain injury protocol applicable to a patient complaining of dizziness and numbness, or a brain injury protocol applicable to an unconscious patient. The sub-protocol may be exactly the same in both brain injury protocols, even though the brain injury protocols are directed to different situations.

The term “brain injury protocol” also encompasses recording and record keeping that accompanies attending to patient 10. When a brain injury protocol calls for a specific sequence of actions, for example, the brain injury protocol may include recording each action taken and documenting how each action was performed. By contrast, when a brain injury protocol calls for a routine or non-specific action, the brain injury protocol might not require recording the details of how the action was performed. Memory 22 may maintain an “event log,” which records actions in the course of attending to patient 10.

In some brain injury protocols, timing of treatment is important. In some cases, a brain injury protocol may not only specify what drugs are to be administered and in what amounts, but also the timing of the dosages. In other cases, a brain injury protocol may specify that a therapy such as cooling therapy be applied for a particular duration. Accordingly, the term “brain injury protocol” also includes schedules and timers for administering treatment.

“Brain injury protocol” further includes modes for presentation of information. System 12 includes one or more output devices 24A-24N (hereinafter 24), by which system 12 present information to an operator. Output devices 24 comprise, for example, a touch screen, a display screen such as a cathode ray tube (CRT) or liquid crystal display (LCD) device, an audible sound generator, a voice synthesizer, a printer or an indicator light. Pursuant to a brain injury protocol, system 12 presents sets of data that are significant to the patient's condition, such as oxygen saturation of blood flowing to the brain, temperature of the body, or electrical activity in the brain. Pursuant to a brain injury protocol, system 12 also presents interrogations to an operator, to which an operator can respond using input device 20.

“Brain injury protocol” further includes references and utilities for carrying out procedures for diagnosis and treatment of a patient. The term “references” includes any information that may assist in attending to the patient, such as formulas or information about medications patient 10 may be taking. The term “utilities” includes any tools that may assist in treating patient 10. An example of a utility may be a calculator that computes dosages of medication, or converts English units to metric units and vice versa.

“Brain injury protocol” also includes the instructions used by a processor such as microprocessor 18 to present information to assist an operator attending patient 10. A protocol may be embodied as a computer-readable medium comprising instructions for a processor. A “computer-readable medium” includes but is not limited to read-only memory, Flash memory and a magnetic or optical storage medium. The medium may comprise instructions for causing a programmable processor to present information in a variety of formats, and to interact with an operator in many ways.

In addition to storing brain injury protocols, memory 22 stores instructions that direct the operation of microprocessor 18. In addition, memory 22 stores information about patient 10, for example, vital signs such as the blood pressure, heart rate and temperature. Memory 22 can also record therapy applied to patient 10, if any, and the response of patient 10 to the therapy. Data stored in memory 22 may be retrieved via output devices 24 or via communication module 26. The data stored in memory 22 may be useful to medical personnel in diagnosing or treating the patient, and result in a more complete medical record for patient 10. The data stored in memory 22 may be useful for showing that established procedures were followed. An operator such as an EMT may use the recorded data in preparing a “run report” that documents the emergency. The recorded data may also be helpful to the regulating authority that establishes protocols when analyzing whether established protocols could be made more effective.

As shown in FIG. 1, communication module 26 can include a wireless communication device, such as a cellular phone. Communication module 26 may further be configured to establish a communications link with a network, including a local network, the Internet, a telephone network or a wireless communication network. Data stored in memory 22 may be transmitted to a remote unit, such as a hospital, via communication module 26. In addition, system 12 may receive updates to brain injury protocols form a remote unit via communication module 26.

In the example of FIG. 1, system 12 also includes a therapy device 28, coupled to system 12 via a therapy device interface 30. Therapy device 28 is any device that can provide therapy to address actual or possible brain injury. In FIG. 1, therapy device 28 comprises a cooling apparatus that cools the head of patient 10 via a headgear. A cooling apparatus provides hypothermic therapy that may be helpful to patients who have suffered strokes.

The invention is not limited to the cooling apparatus shown in FIG. 1, however. In some embodiments of the invention, therapy device 28 may include a set of cooling garments that cool the arms, torso and legs of the patient. In other embodiments of the invention, therapy device 28 includes no cooling garments, but administers some other therapy, such as drug therapy. Administration of thrombolytics, for example, can benefit patients suffering from ischemic stroke. Other examples of therapy devices 28 include oxygen regulators or ventilators that assist the patient with breathing, or automated chest compressors that restore some circulation of blood to the brain following a cardiac failure.

Microprocessor 18 controls therapy device 28 according to the selected brain injury protocol. In the case therapy device 28 includes a cooling garment, microprocessor 18 controls the temperature to which patient 10 will be cooled, and how rapidly the cooling occurs. Microprocessor 18 can further receive feedback from a monitoring device 14 when controlling therapy device 28.

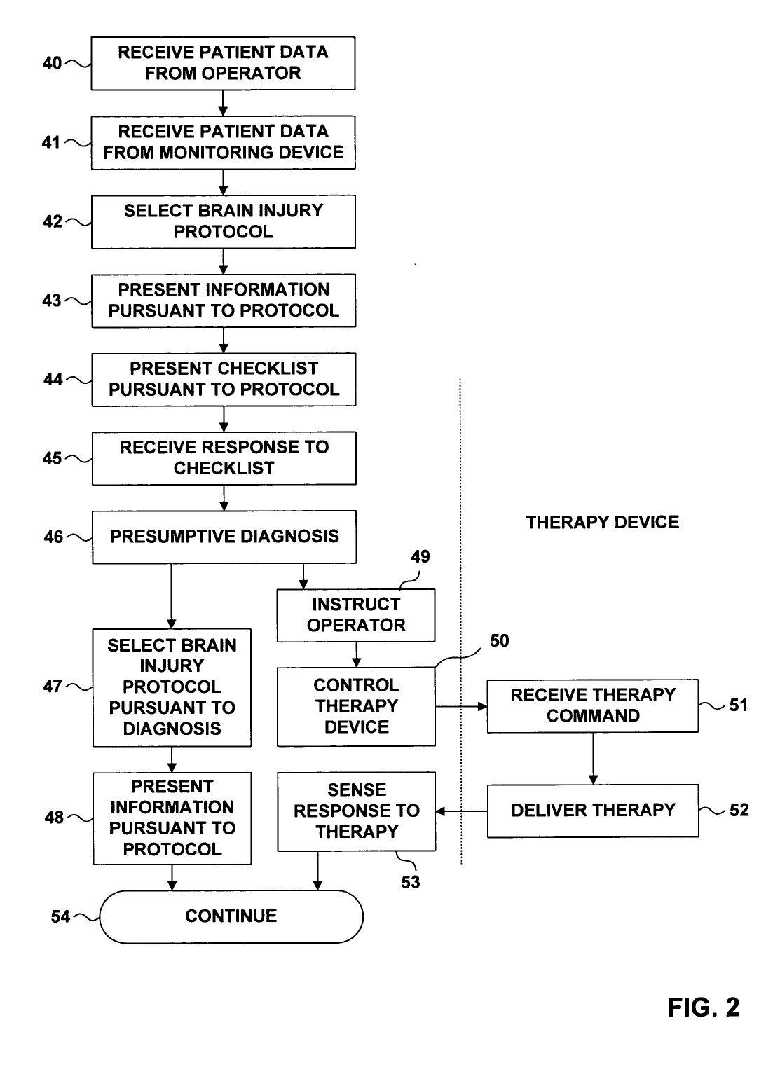

FIG. 2 is a flow diagram illustrating an example of a technique for selection of a protocol, presentation of information pursuant to the selected protocol, and controlling a therapy device pursuant to the selected protocol. A processor in a device, such as microprocessor 18 in system 12, receives patient data. The patient data may be received from any of several sources, such as data entered by an operator via input device 20 (40) or data received by a monitoring device 14 (41) or any combination thereof. The data may pertain to the current physical or mental condition of patient 10, the complaints of patient 10, a medical history, measured aspects of medical significance such as heart rate, body temperature or blood pressure, and the like.

The processor selects a brain injury protocol as a function of the received patient data (42). The selected brain injury protocol may be a stroke protocol, for example, when available data suggests the patient has suffered a stroke. The processor presents information via one or more output devices 24 pursuant to the selected protocol (43). Examples of presented information will be discussed below. The presented information guides the operator attending to the patient.

Evaluation of the condition of patient 10 usually involves application of one or more recognized procedures for assessing the neurological condition, which will be discussed below. The procedures typically employ checklists of signs and responses of patient 10. Accordingly, system 12 presents one or more checklists (44) pursuant to the selected protocol. System 12 also receives the response of the operator to the checklists (45), which represents additional patient data. The responses may be stored in memory 22.

On the basis of the received patient data, the processor may determine a presumptive diagnosis (46). The diagnosis is presumptive in the sense that the diagnosis is premised upon the patient data received, and the diagnosis is generally not intended to supplant a diagnosis that may be made by a physician at a later time.

The processor may select another brain injury protocol as a function of the presumptive diagnosis (47) and present additional information pursuant to the protocol (48). The additional information may include presenting the operator with a task to be performed pursuant to the brain injury protocol. The processor may also prompt the operator to supply an acknowledgement that the task has been performed.

For example, the processor may select an ischemic stroke protocol or a hemorrhagic stroke protocol. Although a diagnosis of ischemic versus hemorrhagic stroke typically requires a hospital visit and specialized equipment such as a CT scan, some patient data may suggest that ischemic stroke is more likely than hemorrhagic stroke, or vice versa. High blood pressure suggests hemorrhagic stroke, for example, while the presence of clot-forming elements such as an artificial heart valve suggests an ischemic stroke. The approaches to ischemic stroke and hemorrhagic stroke are different in several respects, and the protocols reflect the differences.

Presentation of information generally includes presentation of information pertaining to treatment of patient 10. For example, system 12 may notify the operator to administer a dosage of medication, and may further prompt the operator to enter an acknowledgement that administration of the dosage has been accomplished. System 12 may record the administration of the dosage as an event in the event log in memory 22. System 12 may also record other matters pertaining to the dosage, such as the drug administered, the concentration, the mode of administration (such as drip or bolus) and the time of administration. If the drug is one that should be administered at specified intervals according to a protocol, system 12 may also activate a timer that will prompt the operator to administer the drug at those intervals.

When application of therapy device 28 is indicated, system 12 instructs the operator to perform a task, i.e., to apply therapy device 28 (49) to patient 10. System 12 may further prompt the operator to acknowledge that therapy device 28 has been applied. For example, system 12 may instruct the operator to apply cooling garments to patient 10, and to acknowledge when the cooling garments are in place. Memory 22 stores a record that patient 10 received cooling garments as an “event” in an event log. Once therapy device 28 is in place, the processor may control therapy device 28 (50) to deliver therapy to patient 10.

Some therapy devices may include dedicated processors or controllers that regulate the operation of the particular device. A cooling garment, for example, may include controllers that regulate the flow of coolant and air. Such therapy devices receive commands from system processor 12 (51) and deliver therapy according to the therapy commands (52).

The processor monitors the response of patient 10 to therapy (53), by receiving patient data entered by an operator via input device 20 or data received by a monitoring device 14 or any combination thereof. Memory 22 typically records the therapy and patient response as events. In addition, the processor may use the response of patient 10 as feedback used for controlling the therapy. For example, the processor may control a cooling garment to provide rapid cooling to patient 10, and may reduce the cooling rate as a function of the measured temperature of patient 10.

The processor may continue (54) receiving patient data, presenting information and administering therapy. In some cases, the processor may select another brain injury protocol as a function of the received data. Under some circumstances, the processor may discontinue therapy, or give one therapy priority over another. Should the heart of the patient stop while the patient is receiving cooling therapy, for example, the processor would generally give therapies to restart the heart and restore circulation priority over cooling therapies. The processor may suspend cooling and direct the therapy to defibrillation or chest compression.

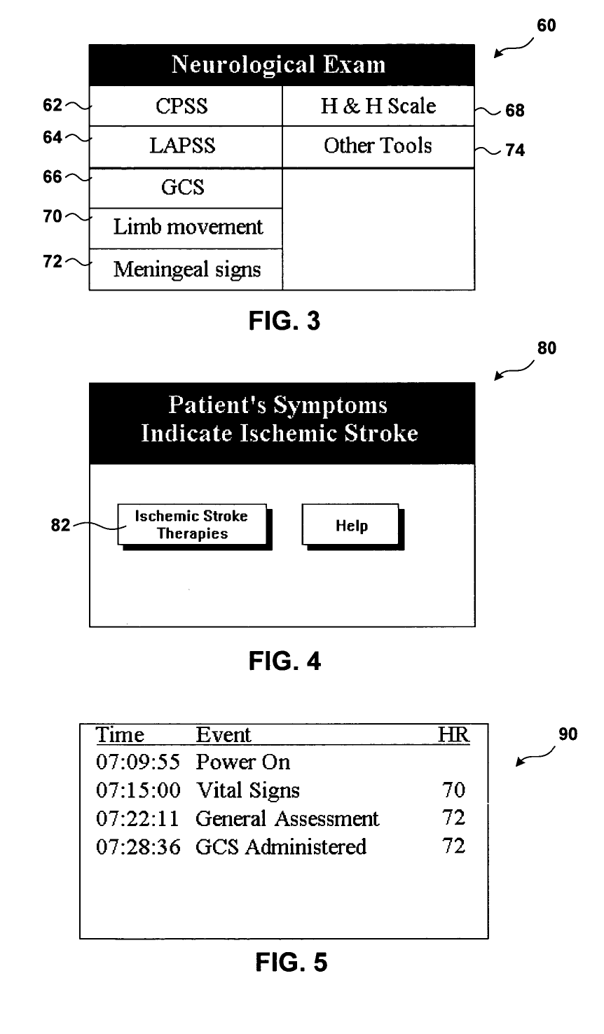

FIG. 3 is an exemplary screen display 60 listing a menu of various possible brain injury protocols supported by a device such as system 12. Protocols displayed in FIG. 3 include one or more protocols for assessment of the neurological condition of patient 10. The operator may make a selection from the menu by using an input device 20, e.g., by touching a touch screen, pointing with a mouse or pressing a button.

There are many recognized procedures for assessing the neurological condition of patient 10. Recognized procedures include the Cincinnati Prehospital Stroke Scale (CPSS) 62, the Los Angeles Prehospital Stroke Screen (LAPSS) 64, the Glasgow Coma Scale (GCS) 66 and the Hunt and Hess Scale for Subarachnoid Hemorrhage (H&H Scale) 68. In addition, a neurological examination of patient 10 may include procedures for checking limb movement 70 or checking for meningeal signs 72.

An operator may apply any or all of the procedures for assessing the neurological condition of patient 10. In some cases, an operator may make a general assessment of the condition of the patient before selecting an examination procedure. As will be discussed below, system 12 may be configured to assist the operator in applying the procedures and recording the results.

FIG. 3 does not show an exclusive list of neurological examination procedures, and many other procedures may be appropriate. Accordingly, screen display 60 lists an “Other Tools” option 74, and the operator may select this option to have access to other neurological examination procedures.

On the basis the response of patient 10 to the neurological examination, system 12 may inform the operator of a presumptive diagnosis. FIG. 4 is an exemplary screen display 80 showing a presumptive diagnosis of ischemic stroke. Screen display 80 may include a prompt 82 to the operator, asking the operator to acknowledge the presumptive diagnosis before proceeding with brain injury protocols that include therapy for ischemic stroke.

FIG. 5 is an exemplary screen display 80 of an event log. An event log may include a report showing actions taken in the course of attending to patient 10. As shown in FIG. 5, an event log may include a description of the action and the time the action was taken. An event log may further include other data associated with the event, such as a monitored heart rate. An event log may be useful for emergency room personnel in a hospital who need to know the course of the pre-hospital treatment. An event log may also be helpful to an operator in preparing a run report.

In addition, an event log may be used to determine whether the operator followed an established protocol, or whether the operator had reason to depart from the established protocol. The effectiveness of a protocol itself may be analyzed by analysis of one or more event logs.

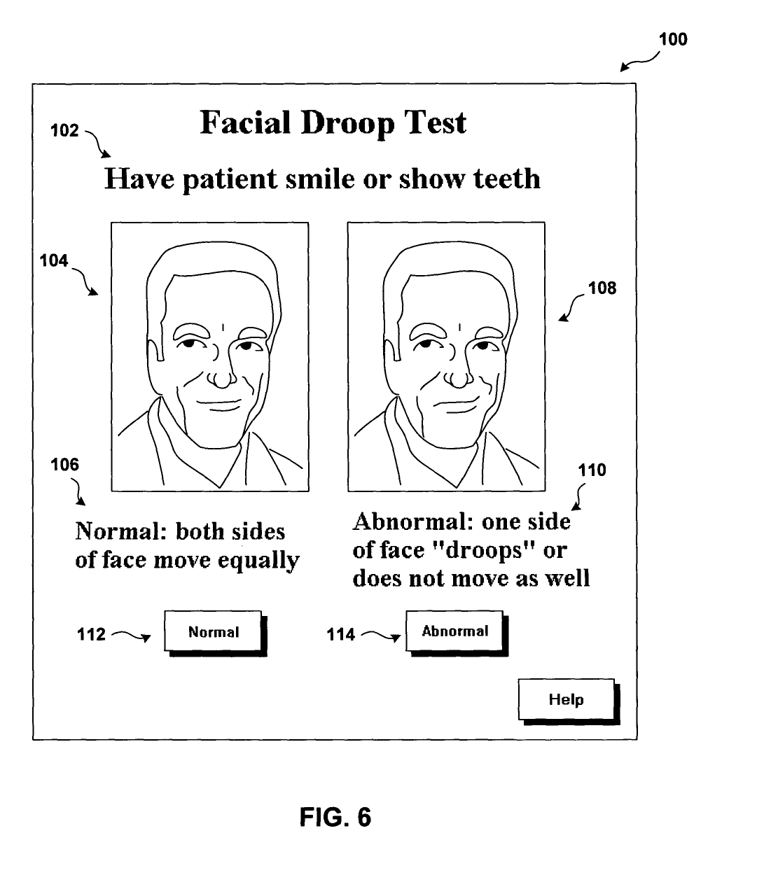

FIG. 6 is an exemplary screen display 100 of a typical test administered as part of a typical examination of a conscious patient. In particular, FIG. 6 illustrates the “Facial Droop Test” that is a part of procedures such as the CPSS.

As shown in FIG. 6, screen display 100 prompts the operator 102 to instruct patient 10 to smile or show his teeth. Screen display 100 includes a graphic 104 showing a normal response, along with a written explanation 106 describing a normal response. Screen display 100 further includes a graphic 108 showing an abnormal response, along with a written explanation 110 describing an abnormal response. In addition, screen display 100 offers the operator a choice of a “Normal” selection 112 and an “Abnormal” selection 114.

System 12 receives the selection by the operator of “Normal” 112 or “Abnormal” 114, and the selection is recorded in memory 22. The response of the patient to the Facial Droop Test may be important in reaching a presumptive diagnosis, because facial droop may be indicative of a stroke.

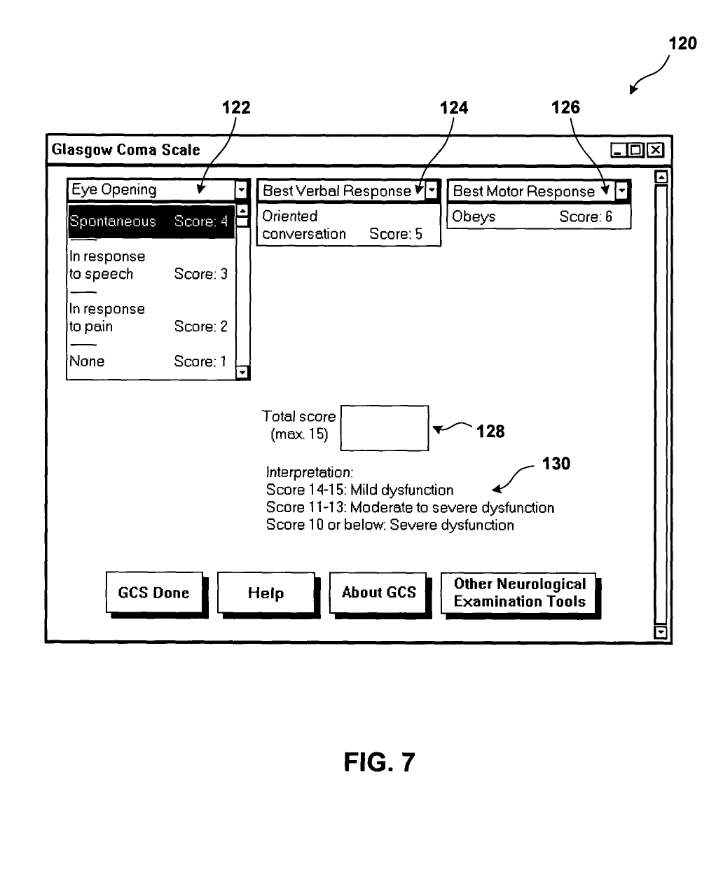

FIG. 7 is an exemplary screen display 120 of a typical test administered as part of a typical examination of a patient who may be conscious, unconscious, or showing dysfunction. In particular, FIG. 7 illustrates the GCS, which evaluates a patient according to criteria of eye opening 122, verbal response 124 and motor response 126. As shown with the eye opening criteria 122, possible responses of patient 10 are assigned a score. In FIG. 7, an operator selects the response of patient 10 in each of the criteria by selecting from a pulldown menu. Screen display 120 automatically shows the score 128 and includes guidelines for interpretation of the score 130. System 12 receives the assessment of the performance of the patient in each of the criteria and records the results in memory 22. The response of the patient to the GCS may be important in reaching a presumptive diagnosis.

The invention may have one or more advantages. Emergency medical personnel may respond to a wide variety of emergencies, and it may be difficult to remember the protocols for all of the situations the personnel may encounter. In addition, there may be many neurological examination procedures, such as stroke scales or other assessment tools, that may be applicable to different situations. The invention helps guide the emergency medical personnel through the established procedures of an applicable protocol, and helps the emergency medical personnel apply the appropriate procedures.

During an emergency rescue operation, emergency medical personnel may lose track of which procedures have been performed and which have not, or the personnel may lose track of time. The invention helps emergency medical personnel keep track of what has been done, and also helps emergency medical personnel keep track of time so that drug dosages or other therapies may be administered at appropriate times.

The invention may be embodied in a medical device such as a defibrillator or a patient monitor that the emergency medical personnel routinely carry. As a result, a device that is routinely transported to emergencies may assist the emergency medical personnel in following an established protocol.

The invention may further assist in recording events related to attending a patient. As noted above, recording events in an event log may be advantageous to the operator attending to the patient, the medical personnel at the hospital and the regulating authority, among others.

The invention may also provide a convenient integration of functions that are traditionally separate. Various embodiments of the invention can collect and record patient data, select one or more brain injury protocols, present information to an operator according to the brain injury protocols, and control one or more therapy devices according to the protocols. The invention supports cooperation among the various functions. Patient data collected from a monitoring device may be used to control a therapy device, for example.

The invention accommodates a variety of brain injury protocols. The invention allows a regulating authority to implement its own local brain injury protocols. In addition, the invention can be adapted for operators of different degrees of training.

The preceding specific embodiments are illustrative of the practice of the invention. Various modifications may be made without departing from the scope of the claims. For example, the invention need not be embodied in a medical device such as defibrillator or medical monitor. The invention may be embodied in a stand-alone device that provides no diagnosis, monitoring or therapy. The device may be small and easily portable, but the invention is not limited to application with small, portable devices. Nor is the invention limited to medical devices. The invention may also be embodied in a device that performs functions other than medical functions, such as a personal digital assistant or a cellular telephone.

The invention has been described in the context of use by early responders to medical emergencies, such as EMTs. The invention is not limited to use by EMTs or other responders in the field, however. On the contrary, a physician diagnosing the patient may apply the same stroke scales or other procedures to assess the neurological condition of the patient, and may therefore benefit from the invention as well. Embodiments of the invention may be used in a hospital environment. In addition, embodiments of the invention may be used by lay responders having less training than EMTs, such as law enforcement or security personnel.

The invention may be embodied as a computer-readable medium that includes instructions for causing a programmable processor to carry out the methods described above. A “computer-readable medium” includes but is not limited to read-only memory, Flash memory and a magnetic or optical storage medium. The instructions may be implemented as one or more software modules, which may be executed by themselves or in combination with other software.

The instructions and the media are not necessarily associated with any particular computer or other apparatus, but may be carried out by various general-purpose or specialized machines. The instructions may be distributed among two or more media and may be executed by two or more machines. The machines may be coupled to one another directly, or may be coupled through a network, such as a local access network (LAN), or a global network such as the Internet.

The invention may also be embodied as one or more devices that include logic circuitry to carry out the functions or methods as described herein. The logic circuitry may include a processor that may be programmable for a general purpose or may be dedicated, such as microcontroller, a microprocessor, a Digital Signal Processor (DSP), Application Specific Integrated Circuit (ASIC), and the like. These and other embodiments are within the scope of the following claims.

1. A method comprising:
receiving data concerning a patient; and
selecting a brain injury protocol as a function of the data.

2. The method of claim 1, further comprising receiving the data from an operator.

3. The method of claim 1, further comprising receiving the data from a monitoring device.

4. The method of claim 3, in which the data from the monitoring device comprises at least one of a heart rate, a pulse, a blood pressure, a carbon dioxide concentration, a blood oxygen concentration, a respiration, a blood velocity, an EEG, an ECG, and a body temperature.

5. The method of claim 1, in which the data comprise at least one of a physical condition of the patient, a mental condition of the patient and a complaint from the patient.

6. The method of claim 1, in which the brain injury protocol comprises one of an ischemic stroke protocol and a hemorrhagic stroke protocol.

7. The method of claim 1, further comprising controlling a therapy device according to the brain injury protocol.

8. The method of claim 7, in which the therapy device comprises one of a cooling device and a drug delivery device.

9. The method of claim 1, further comprising:
presenting an operator with a task to be performed pursuant to the brain injury protocol; and
receiving an acknowledgement from the operator that the task has been performed.

10. The method of claim 1, further comprising storing in an event log at least one of an action taken in the course of attending to the patient, a reception of the data from a monitoring device and a therapy delivered by a therapy device.

11. The method of claim 10, further comprising transmitting the event log to a remote unit.

12. The method of claim 1, further comprising:
presenting a checklist to an operator pursuant to the brain injury protocol, the checklist requesting entry of additional data concerning the patient; and
receiving the additional data from the operator.

13. The method of claim 12, further comprising controlling a therapy device as a function of the additional data.

14. The method of claim 1, further comprising determining a presumptive diagnosis as a function of the data.

15. A method comprising:
presenting a checklist to an operator, the checklist requesting entry of data concerning a neurological condition of a patient;
receiving the data from the operator;
receiving additional data concerning the patient from a monitoring device; and
selecting a brain injury protocol as a function of the data.

16. The method of claim 15, in which the checklist comprises at least one of a Cincinnati Prehospital Stroke Scale, a Los Angeles Prehospital Stroke Screen, a Glasgow Coma Scale and a Hunt and Hess Scale for Subarachnoid Hemorrhage.

17. The method of claim 15, further comprising determining a presumptive diagnosis as a function of the data.

18. The method of claim 15, in which the data from the monitoring device comprises at least one of a heart rate, a pulse, a blood pressure, a carbon dioxide concentration, a blood oxygen concentration, a respiration, a blood velocity, an EEG, an ECG, and a body temperature.

19. The method of claim 15, further comprising controlling a therapy device according to the brain injury protocol.

20. The method of claim 15, further comprising storing in an event log at least one of an action taken in the course of attending to the patient, a reception of the data from a monitoring device and a therapy delivered by a therapy device.

21. A system comprising:
a monitoring device to monitor a medical condition of a patient and to generate data concerning the patient; and
a processor to receive the data from the monitoring device and to select a brain injury protocol as a function of the data.

22. The system of claim 21, further comprising a therapy device to provide brain injury therapy to the patient.

23. The system of claim 22, in which the processor is configured to control the therapy device as a function of the data.

24. The system of claim 22, in which the processor is configured to control the therapy device as a function of the brain injury protocol.

25. The system of claim 22, in which the therapy device comprises one of a cooling device and a drug delivery device.

26. The system of claim 21, further comprising an input device to receive additional data concerning the patient from an operator.

27. The system of claim 26, in which the input device comprises at least one of a button, a keyboard, a touch screen, a voice recognition module and a pointing tool.

28. The system of claim 21, further comprising an output device to present brain information pursuant to the brain injury protocol.

29. The system of claim 28, in which the output device comprises at least one of a touch screen, a display screen, an audible sound generator, a voice synthesizer, a printer and an indicator light.

30. The system of claim 21, further comprising memory to store at least one of a brain injury protocol and a datum concerning the patient.

31. The system of claim 30, further comprising a communication module to transmit data stored in the memory to a remote unit.

32. The system of claim 21, in which the monitoring device comprises at least one of a heart rate monitor, a pulse monitor, a blood pressure monitor, a carbon dioxide concentration sensor, a blood oxygen concentration sensor, a blood velocity sensor, an EEG monitor, an ECG monitor, and a body temperature sensor.

33. A computer-readable medium comprising instructions for causing a programmable processor to:
receive data concerning a patient; and
select a brain injury protocol as a function of the data.

34. The medium of claim 33, the instructions further causing the processor to receiving the data from at least one of an operator and a monitoring device.

35. The medium of claim 33, the instructions further causing the processor to control a therapy device according to the brain injury protocol.

36. The medium of claim 33, the instructions further causing the processor to store in an event log at least one of an action taken in the course of attending to the patient, a reception of the data from a monitoring device and a therapy delivered by a therapy device.

37. The medium of claim 33, the instructions further causing the processor to determine a presumptive diagnosis as a function of the data.

38. A computer-readable medium comprising instructions for causing a programmable processor to:
present a checklist to an operator, the checklist requesting entry of data concerning a neurological condition of a patient;
receive the data from the operator;
receive additional data concerning the patient from a monitoring device; and
select a brain injury protocol as a function of the data.

39. The medium of claim 38, in which the checklist comprises at least one of a Cincinnati Prehospital Stroke Scale, a Los Angeles Prehospital Stroke Screen, a Glasgow Coma Scale and a Hunt and Hess Scale for Subarachnoid Hemorrhage.

40. The medium of claim 38, the instructions further causing the processor to control a therapy device according to the brain injury protocol.

41. The medium of claim 38, the instructions further causing the processor to determine a presumptive diagnosis as a function of the data.
